(19) United States
(12) Patent Application Publication
(10) Pub. No.: US 2006/0025380 A1
(43) Pub. Date: Feb. 2, 2006

(54) Formulations for non-parenteral use including hydrophobic cyclodextrins

(75) Inventors: Thorsteinn Thorsteinsson, Minooka, IL (US); Matthew Duan, Lisle, IL (US); Robert P. O'Fee, New Hope, PA (US)

Correspondence Address: Jeffrey M. Hoster;Jeffrey M. Hoster, Ltd., 13 Woodland Drive, Lemont, IL, 60439, US

(73) Assignee: deCODE Chemistry Inc

(21) Appl. No.: 11/127,400

(22) Filed: Oct. 18, 2005

Related U.S. Application Data
(60) Provisional application No. 60/571,253, filed on May 14, 2004.

(51) Int. Cl. A61K 31/724; A61K 31/4172; A61K 31/405; A61K 31/401; A61K 31/198

(52) U.S. Cl. 514/58; 514/419; 514/400; 514/423; 514/561; 514/567; 514/562; 514/565; 514/566

(57) ABSTRACT

A novel system for non-parenteral formulations comprising cyclodextrins is disclosed. The system includes hydrophobic cyclodextrins and amino acids and homo- or co-polymers thereof. The cyclodextrins and amino acids form a complex with pharmaceutical and other ingredients to achieve greatly improved solubility and/or enhance stability. The complexes can be used for delivery to mammals in a wide variety of non-parenteral formulations.

### BACKGROUND OF THE INVENTION

1. Field of the Invention

The present invention relates to the field of delivery systems for pharmaceutical and other products such as foods, beverages, nutritional products, cosmetics, and agrochemicals. More specifically, it relates to the field of such products in which the compound of interest is delivered via a transdermal, ophthalmic, intranasal, sublingual, oral or other non-parenteral delivery system that makes use of cyclodextrins and/or other components.

2. Related Background Art

Formulation of pharmaceutical dosage forms is frequently hampered by the poor aqueous solubility and stability of the drugs, which in turn can severely limit their therapeutic application. Also, the slow dissolution of solid state drug formulations and the side-effects of some drugs result from their poor aqueous solubility. Drug degradation products, formed in the pharmaceutical dosage forms, can also result in severe side-effects. Increasing drug solubility and stability through appropriate formulations can lead to increased therapeutic efficiency of the drug. Various methods have been used to increase the solubility and stability of drugs, such as the use of organic solvents, emulsions, liposomes and micelles, adjustments of pH and the dielectric constant of the solvent system, chemical modifications, and complexation of the drugs with appropriate complexing agents, e.g. cyclodextrins.

Non-pharmaceutical products are also dependent on formulation solubility and stability. Many foods, beverages, cosmetics and nutritional products include ingredients which are relatively insoluble. Solubility is often important for taste, palatability or appearance reasons. For products such as agrochemicals, efficacy often depends on the effective solubility of the active ingredients to ensure that proper dispersion of the active ingredient occurs.

Cyclodextrins were first isolated by Villiers in 1891 as a digest of Bacillus amylobacter on potato starch [see A. Villiers: Sur la fermentation de la fecule par l'action du ferment butyrique. C. R. Acad. Sci., 112, 536-538 (1891)], but the foundations of cyclodextrin chemistry were laid down by Schardinger in the period 1903-1911 [see, for example, F. Schardinger: Uber thermophile Bacterien aus verschiedenen Speisen and Milch, sowie uber einige Umsetzungsproducte darselben in kohlenhydrathaltigen Nahrlosungen, darunter krystallisierte Polysaccharide (Dextrine) aus Starke, Z. Unters. Nahr. GenuBm., 6, 865-880 (1903)] and much of the older literature refers to cyclodextrins as Schardinger's dextrins. Until 1970, only small amounts of cyclodextrins could be produced in the laboratory and the high production cost prevented the usage of cyclodextrins in industry. In recent years, dramatic improvements in cyclodextrin production and purification have been achieved and the cyclodextrins have become much cheaper. This has made industrial application of cyclodextrins possible.

Cyclodextrins are cyclic oligosaccharides with hydroxyl groups on the outer surface and a void cavity in the center. Their outer surface is hydrophilic, and therefore they are usually soluble in water, but the cavity has a lipophilic character. The most common cyclodextrins are alpha.-cyclodextrin, beta.-cyclodextrin and gamma.-cyclodextrin, consisting of 6, 7 and 8 alpha.-1,4-linked glucose units, respectively. The number of these units determines the size of the cavity.

Cyclodextrins are capable of forming inclusion complexes with a wide variety of hydrophobic molecules by taking up a whole molecule, or some part of it, into the cavity. The stability of the complex formed depends on how well the guest molecule fits into the cyclodextrin cavity.

Common cyclodextrin derivatives are formed by alkylation (e.g. methyl- and ethyl-.beta.-cyclodextrin) or hydroxyalkylation of the hydroxyl groups (e.g. hydroxypropyl- and hydroxyethyl-derivatives of alpha.-, beta.-, and gamma.-cyclodextrin) or by substituting the primary hydroxyl groups with saccharides (e.g. glucosyl- and maltosyl-beta.-cyclodextrin). Hydroxypropyl-beta.-cyclodextrin and its preparation by propylene oxide addition to beta.-cyclodextrin, and hydroxyethyl beta.-cyclodextrin and its preparation by ethylene oxide addition to beta.-cyclodextrin, were described in a patent of Gramera et al. (U.S. Pat. No. 3,459,731, issued August 1969) over 20 years ago. For a comprehensive review of cyclodextrins see Cyclodextrins and their industrial uses, editor Dominique Duchene, Editions Sante, Paris, 1987. For a more recent overview, see J. Szejtli: Cyclodextrins in drug formulations: Part 1, Pharm. Techn. Int. 3(2), 15-22 (1991); and J. Szejtli: Cyclodextrins in drug formulations: Part II, Pharm. Techn. Int. 3(3), 16-24 (1991).

Numerous reports have been published with respect to the solubilizing effects of cyclodextrins. The general procedure described in these reports for preparing aqueous cyclodextrin solutions containing various drugs is as follows. An excess amount of the drug is added to an aqueous cyclodextrin solution and the suspension formed is agitated for up to one week at room temperature. Then the suspension is filtered or centrifuged to form a clear drug-cyclodextrin complex solution. For the preparation of solid formulations of the drug-cyclodextrin complex, the water is removed from the aqueous drug-cyclodextrin complex solution by evaporation in a rotation evaporator, in a spray dryer or by lyophilization.

Pitha (Josef Pitha: Administration of sex hormones in the form of hydrophilic cyclodextrin derivatives, U.S. Pat. No. 4,596,795, issued Jun. 24, 1986) describes inclusion complexes of sex hormones, particularly testosterone, progesterone, and estradiol, with specific cyclodextrins, preferably hydroxypropyl-.beta.-cyclodextrin and poly-.beta.-cyclodextrin. The complexes enable the sex hormones to be successfully delivered to the systemic circulation via the sublingual or buccal route. In another patent (Josef Pitha: Pharmaceutical preparations containing cyclodextrin derivatives, U.S. Pat. No. 4,727,064, issued Feb. 23, 1988) Pitha describes formulations of a number of drugs with various cyclodextrin derivatives, mainly hydroxypropyl-beta.-cyclodextrin but also hydroxypropyl-gamma.-cyclodextrin. In a series of patents (N. S. Bodor: Improvements in redox systems for brain-targeted drug delivery, U.S. Pat. No. 5,002,935, issued Mar. 26, 1991; N. S. Bodor: Pharmaceutical formulations for parenteral use, U.S. Pat. No. 4,983,586, issued Jan. 8, 1991; N. S. Bodor: Redox systems for brain-targeted drug delivery, U.S. Pat. No. 5,017,566, issued May 21, 1991; and N. S. Bodor: Pharmaceutical formulations for parenteral use, U.S. Pat. No. 5,024,998, issued Jun. 18, 1991), Bodor describes formulations of a number of drugs with hydroxypropyl, hydroxyethyl, glucosyl, maltosyl and maltotriosyl derivatives of beta.- and gamma.-cyclodextrin. Also, Brauns and Muller (U. Brauns and B. W. W. Muller: Pharmazeutische Praparate von in Wasser schwerloslichen oder instabilen Arznelstoffen und Verfahren zu Ihrer Herstellung, European Patent No. 0 149 197 B1 dated Mar. 21, 1990) have described formulations of drugs with various beta.-cyclodextrin derivatives, mainly hydroxypropyl-beta.-cyclodextrin. The solubilizing and stabilizing effects of hydroxypropyl-beta.-cyclodextrin on drugs have been reviewed by T. Loftsson, M. E. Brewster, H. Derendorf and N. Bodor: 2-Hydroxypropyl-beta.-cyclodextrin: Properties and usage in pharmaceutical formulations. Pharm. Ztg. Wiss. 4/136: 5-10 (1991).

Methods of preparing drug-cyclodextrin complexes have been described by Hirayama and Uekama [F. Hirayama and K. Uekama: Methods of investigating and preparing inclusion compounds. In: D. Ducheene (editor), Cyclodextrins and their industrial uses. Editions de Sante, Paris, 1987, pp. 133-172]. In solution, the drug-cyclodextrin complexes are prepared by the simple method described above and the complexation evaluated by determination of stability constants by a solubility method, a kinetic method, a spectroscopic method or some other analytical method. On a laboratory scale, solid drug-cyclodextrin complexes are usually formed by lyophilization of drug-cyclodextrin complex solution, but on an industrial scale, other methods are also used such as the kneading method, spray-drying, co-precipitation, neutralization and grinding methods. In none of these methods are water-soluble pharmaceutical polymers, or other polymers in general, used for enhancing the drug-cyclodextrin complexation.

There are few samples of formation of drug-cyclodextrin complexes by heating. Hassan et al., Int. J. Pharm. 58, 19-24 (1990), prepared a famotidine-beta.-cyclodextrin complex by adding the drug to aqueous beta.-cyclodextrin solution, heating the mixture under reflux for 1 hour and then stirring it at room temperature for 5 days. The solution which formed was concentrated by evaporation under vacuum and the precipitate which formed was filtered and dried under vacuum at 50 degrees C. In a series of articles, Nakai et al. describe how they make cyclodextrin inclusion complexes by heating ground mixtures of physical mixtures to 60 degrees to 130 degrees C. in sealed containers. See Nakai et al., Chem. Pharm. Bull. 35(11), 4609-4615 (1987); Nakai et al., Chem. Pharm. Bull. 37(4), 1055-1058 (1989); Nakai et al., Chem. Pharm. Bull. 38(3), 728-732 (1990); Nakai et al., Chem. Pharm. Bull. 38(5), 1345-1348 (1990); and Nakai et al., Chem. Pharm. Bull. 39(6), 1532-1535 (1991). Finally, Schmidt and Maier [E. Schmidt and H. G. Maier: Thermostabile Bindung von Aromastoffen an Starke. Teil 2: Bindung von Menthol durch Autoklavieren, Starch/Starke, 39(6), 203-207 (1987)] describe formation of thermostable binding of menthol to various types of starches, including beta.-cyclodextrin, by autoclaving. In none of the above mentioned articles are starches, or other polymers, used to enhance complexation of drugs by cyclodextrins.

Due to the negative enthalpy of cyclodextrin complexation, the solubility enhancement of drugs by aqueous cyclodextrin solutions is generally larger at low temperature than at high temperature [T. Loftsson and N. Bodor: Effects of 2-hydroxypropyl-.beta.-cyclodextrin on the aqueous solubility of drugs and transdermal delivery of 17.beta.-estradiol, Acta Pharm. Nord., 1(4), 185-193 (1989)]. Also, additives such as sodium chloride, buffer salts, surfactants and organic solvents (e.g. ethanol) usually reduce the solubilizing effects of cyclodextrins.

Recently, attempts have been made to improve the complexation and solubilizing and stabilizing effects of cyclodextrins. One approach has been to chemically modify the cyclodextrin molecule. For example, U.S. Pat. No. 5,904,929 to Uekama teaches the use of acylated forms of cyclodextrin to improve solubility. U.S. Pat. No. 6,407,079 teaches the use of cyclodextrin ethers or esters for the same purpose. Two recent patents [T. Loftsson: Cyclodextrin/drug Complexation, U.S. Pat. No. 5,324,718, issued Jun. 28, 1994, and T. Loftsson: Cyclodextrin Complexation, U.S. Pat. No. 5,472,954, issued Dec. 5, 1995] describe complexes using a pharmacologically inactive water-soluble polymer. In the invention detailed in the patents, a water soluble polymer such as a cellulose derivative is co-solubilized with cyclodextrin. An active ingredient such as a drug is then added to the soluble medium, and water is removed. The resulting products have been found to improve solubility and stability. While the above-described patents are effective for many applications, there are certain active compounds having extremely low solubility (<0.1 μg/mL) requiring additional improvements in solubility. Further, the percentage improvement in solubility does not always justify the high cost involved in producing the acylated, ester, or ether forms of cyclodextrin.

Generally, in conventional attempts to improve solubility of drug and other products by use of cyclodextrins, the cyclodextrins have been selected on the basis of their hydrophilicity. Therefore, cyclodextrins such as 2-hydroxypropyl cyclodextrin have been preferred for use in the improvement of solubility. However, use of such cyclodextrins does not result in certain benefits of other less hydrophilic cyclodextrins. Such less hydrophilic cyclodextrins include randomly methylated cyclodextrins and other cyclodextrins that have a more hydrophobic outer surface than the natural cyclodextrins. These cyclodextrins typically have lower molecular weights than the more hydrophilic derivatives such as hydroxy-alkylated or sulfobutylated cyclodextrins. This is critical to controlling the bulk mass of the overall formulation. In many cases, these cyclodextrins have solubilities comparable to their hydrophilic counterparts. Finally, from a commercial point of view, in many cases the hydrophobic cyclodextrins are less expensive than the comparable grade of hydroxy-alkylated or sulfobutylated cyclodextrin. This can lead to significant cost savings.

### SUMMARY OF THE INVENTION

The present invention is a delivery system for pharmaceutical ingredients and other products requiring improved solubility. A complex is formed by use of a hydrophobic cyclodextrin together with an amino acid, amino acid analog, and/or homo- or co-polymers of amino acids. The resulting composition can be used to improve solubility and resulting delivery of products such as pharmaceuticals, foods and beverages, nutritional products, agrochemicals, and cosmetics. These active ingredients can be added to the complex. For pharmaceutical products, the resulting composition can be delivered by any non-parenteral means. For other categories of products, the resulting composition will have improved solubility, resulting in increased flexibility for product use.

### BRIEF DESCRIPTION OF THE DRAWINGS

FIG. 1 shows the effect of the addition of amino acids on the solubility of compositions including cyclodextrin, alone or in combination with other substances, further including as an active pharmaceutical substance trichlocarban.

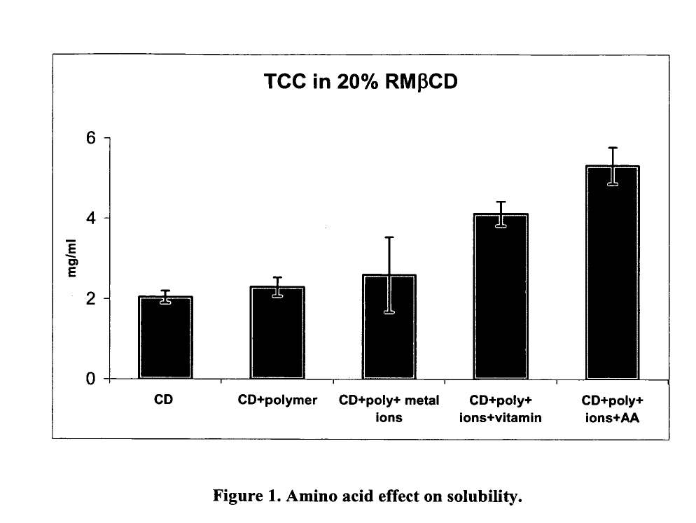

FIG. 2 shows the solubility effect attributable to amino acids in compositions including cyclodextrin, alone or in combination with other substances, including metal ions.

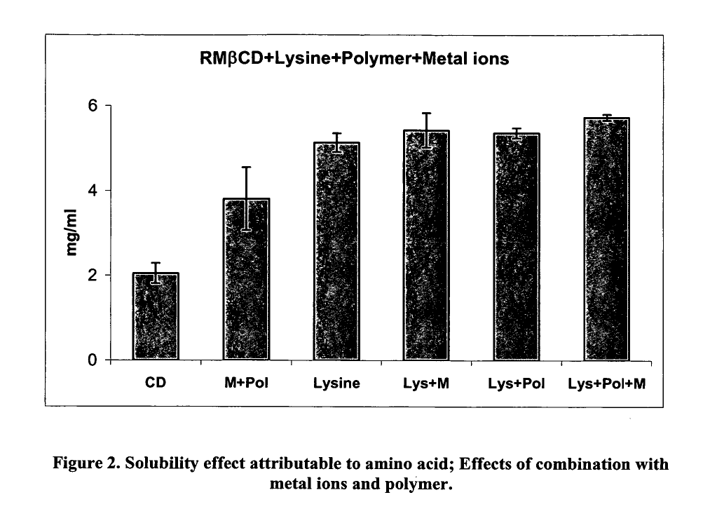

### DETAILED DESCRIPTION OF THE PREFERRED EMBODIMENT

In a preferred embodiment of the present invention, a composition is formed by use of a hydrophobic cyclodextrin together with an amino acid, and/or amino acid analogs, and/or homo-or co-polymers of amino acids, including, for example, di, tri and tetra-peptides of one or more amino acids. Following formation of the composition, an active ingredient such as a pharmaceutical product is added to the medium. The composition preferably also includes at least one component selected from the group comprising metal ions and water soluble polymers.

Cyclodextrins selected for use in the present invention include those selected from the following:

Methyl β-cyclodextrin, dimethyl β-cyclodextrin, trimethyl β-cyclodextrin, randomly methylated β-cyclodextrin, randomly methylated a-cyclodextrin, randomly methylated d-cyclodextrin, a, β, d-cyclodextrins, and other alkylated cyclodextrins.

Suitable amino acids for use herein include Alanine, Isoleucine, Leucine, Methionine, Phenylalanine, Proline, Tryptophan, Valine, Asparagine, Cysteine, Glutamine, Glycine, Serine, Threonine, Tyrosine, Aspartic Acid, Glutamic Acid, Arginine, Hystidine, or Lysine. The amino acids may be in either the d or 1 configuration or include racemic mixtures, salts, or other derivatives thereof. Mixtures of amino acids may also be used.

In general, the amino acids used shall have (in any enantiomeric configuration or racemic mixture thereof) at least one amino group and at least one carboxyl group wherein at least one amino group and at least one carboxyl group are separated by at least one carbon atom.

Particularly preferred amino acids include Lysine, Arginine, Hystidine, Aspartic Acid, Glutamic Acid and Serine. Homo- or co-polymers of such amino acids may also be used. Such homo- or co-polymers include, but are not limited to polylysine and polyarginine. Analogs of amino acids may also be used. Examples of such analogs include, but are not limited to, adipic acid, pipecolinic acid and ornithine. The analogs may be in any isomeric form or racemic or other mixtures thereof.

The ratio of cyclodextrin to amino acid (or homo- or co-polymer of amino acid) is 20:1 to 1:1, more preferably 10:1 to 2:1, and most preferably 10:1 to 5:1.

While not wishing to be bound by theory, it appears that the amino acid or homo- or co-polymer interacts with the cyclodextrin on a non-inclusion basis. That is, the amino acid or homo- or co-polymer does not physically enter the ring structure of the cyclodextrin. Instead, it appears to remain outside the ring structure, providing support to the stability of the structure and permitting the drug to more easily enter the ring structure, which leads to the enhanced solubility.

Other components can be used in the formation of the composition. Because of the unique characteristics of cyclodextrins, the complexes formed can include ternary, quaternary or pentanery complexes. Thus, the additional components can include metal ions, present in the form of +2 or +3 ions. Preferred metal ions include Mg, Fe and Zn. The ions are typically added to the complex simultaneous with cyclodextrin. If used, the molar concentration of the metal ions is preferably about 10 mM to about 200 mM. More preferably, the metal ion is added in a molar concentration of from about 50 mM to about 100 mM.

Other non-active ingredients can be added to the complex in order to provide additional stability or other desired characteristics such as viscosity. Such ingredients may include polymers, vitamins, or gelatin. The amounts of such ingredients are dependent on the specific ingredient and desired usage. For example, if polymer is added, it is preferably added in an amount of from about 0.1% (w/v) to about 0.5% (w/v), based on the amount of cyclodextrin in the complex.

While the inventive complex improves solubility of many pharmaceutical compounds, the inventive complex is especially preferred for use with active pharmaceutical compounds that have low solubility. Solubility is often dependent on the specific dosage form used and specific delivery conditions such as temperature and pH. The use of the present invention is especially preferred with pharmaceutical ingredients such as trichlocarban and camptothecin, and other pharmaceutical ingredients sharing structural similarities thereto. Additionally, pharmaceutical ingredients can be incorporated in the composition according to the invention, including water-soluble and water-sparingly soluble ingredients. In particular, the present invention can be used for administration of pharmaceutical ingredients that are trans-mucosally or transdermally administered, for example, nonsteroidal antirheumatic agents, steroids, cardiac glycosides, benzodiazepine derivatives, benzimidazole derivatives, piperidine derivatives, piperazine derivatives, imidazole derivatives and triazole derivatives. Benzimidazole derivatives that can be used with the inventive composition include but are not limited to thiabendazole, fuberidazole, oxibendazole, parbendazole, cambendazole, mebendazole, fenbendazole, flubendazole, albendazole, oxfendazole, nocodazole and astemizole. Further, as suitable piperidine derivatives, bruspirilen, bimotide, penfluridol, loperamide, ketanserin, levocabastine, cisapride, altanserin and ritanserin are exemplary pharmaceutical ingredients. Piperazine derivatives include but are not limited to lidoflazine, flunarzine, mianserin, oxatomide, miofurazine and cinnarizine. Further, the following imidazole derivatives are suitable for use with the present invention: metronidazole, ornidazole, ipronidazole, tindazole, isoconazole, nimorazole, primamide, methiamide, metomidate, enilconazole, etomidate, econazole, clotrimazole, garnidazole, cimetidine, docodazole, sulconazole, parconazole, orconazole, butoconazole, triadiminol, tioconazole, parconazole, fluotrimazole, ketoconazole, oxyconazole, rombazole, bifonazole, oxcimetidine, fenticonazole and tabrazole. Triazoles and nitrous oxide derivatives may also be suitable for use with the present invention.

The following classes of drugs can also be used with the inventive composition:

analgesic and anti-inflammatory drugs such as acetylsalicylic acid, sodium diclofenac, ibuprofen, indomethacin, ketoprofen, sodium meclofenamate, mefenamic acid, sodium naproxen, paracetamol, piroxicam and sodium tolmetin;

anti-arrhythmic drugs such as procainamide HCl, qunidine sulphate and verapamil HCl;

antibacterial agents such as amoxicillin, ampicillin, benzathine penicillin, benzylpenicillin, cefaclor, cefadroxiI, cephalexin, chloramphenicol, ciprofloxacin, clavulanic acid, clindamycin HCl, doxycycline HCl, erythromycin, sodium flucloxacillin, kanamycin sulphate, lincomycin HCl, minocycline HCl, sodium nafcillin, nalidixic acid, neomycin, norfloxacin, ofloxacin, oxacillin, and potassium phenoxymethyl-penicillin;

anti-coagulants such as warfarin;

antidepressants such as amitriptyline HCl, amoxapine, butriptyline HCl, clomipramine HCl, desipramine HCl, dothiepin HCl, doxepin HCl, fluoxetine, gepirone, imipramine, lithium carbonate, mianserin HCl, milnacipran, nortriptyline HCl and paroxetine HCl;

anti-diabetic drugs such as glibenclamide;

antifungal agents such as amphotericin, clotrimazole, econazole, fluconazole, flucytosine, griseofulvin, itraconazole, ketoconazole, miconazole nitrate and nystatin;

antihistamines such as astemizole, cinnarizine, cyproheptadine HCl, flunarizine, oxatomide, promethazine and terfenadine;

anti-hypertensive drugs such as captopril, enalapril, ketanserin, lisinopril, minoxidil, prazosin HCl, ramipril and reserpine;

anti-muscarinic agents such as atropine sulphate and hyoscine;

antiviral drugs such as acyclovir, AZT, ddC, ddl, ganciclovir, loviride, tivirapine, 3TC, delavirdine, indinavir, nelfinavir, calanolide-A, ritonavir and saquinavir;

sedating agents such as alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, selfotel, seroquel, sulpiride, temazepam, thiothixene, triazolam, trifluperidol and ziprasidone;

anti-stroke agents such as lubeluzole, lubeluzole oxide, riluzole, aptiganel, eliprodil and remacemide;

anti-migraine drugs such as alniditan and sumatriptan;

beta-adrenoreptor blocking agents such as atenolol, carvedilol, metoprolol, nebivolol and propranolol;

cardiac inotropic agents such as digitoxin, digoxin and milrinone;

corticosteroids such as beclomethansone dipropionate, betamethasone, dexamethasone, hydrocortisone, methylprednisolone, prednisolone, prednisone and triamcinolone;

disinfectants such as chlorhexidine;

diuretics such as acetazolamide, frusemide, hydrochlorothiazide and isosorbide;

anti-Parkinsonian drugs such as bromocryptine mesylate, levodopa and selegiline HCl;

enzymes or essential oils such as anethole, anise oil, caraway, cardamom, cassia oil, cinelole, cinnamon oil, clove oil, coriander oil, dementholised mint oil, dill oil, eucalyptus oil, eugenol, ginger, lemon oil, mustard oil, neroli oil, nutmeg oil, orange oil, peppermint, sage, spearmint, terpineol and thyme;

gastro-intestinal agents such as cimetidine, cisapride, clebopride, diphenoxylate HCl, domperidone, famotidine, lansoprazole, loperamide HCl, loperamide oxide, mesalazine, metoclopramide HCl, mosapride, olsalazine, omeprazole, ranitidine, rabeprazole, ridogrel and sulphasalazine;

haemostatics such as aminocaproic acid;

lipid regulating agents such as lovastatin, pravastatin, probucol and simvastatin;

local anesthetics such as benzocaine and lidocaine;

opioid analgesics such as buprenorphine HCl, codeine, dextromoramide and dihydrocodeine;

parasympathomimetics such as galanthamine, neostigmine, physostymine, tacrine, donepezil, ENA 713 (exelon) and xanomeline; and

vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate.

The inventive composition can be used in a wide range of non-parenteral dosage forms, including but not limited to: transdermal and dermal patches and creams, eye drops, syrups such as cough syrups, mouthwash, toothpaste, cosmetics, soaps and detergents with active ingredients. Other forms may include pump sprays, sublingual tablets, sublingual films, quick-dissolve tablets, quick-dissolve films, chewing gums, lozenges, and nanocrystals.

The inventive composition may have uses outside the pharmaceutical ingredient field. For example, the inventive composition may be especially useful with food products, such as carbonated soft drinks and powdered soft drinks. Many low solubility ingredients may add benefits to these products, but require means to improve solubility. Similarly, powdered nutritional supplements to be added to beverages often require improved solubility to be effectively dissolved when such powders are added. Other types of products include children's oral electrolyte maintenance solutions, oral vitamin products, and similar over the counter liquid and syrup products.

One additional benefit of the inventive compositions is found with products having a taste component, for example, products taken orally or intranasally. Many of the amino acids suitable for use with the present invention also have taste modification benefits at or below the taste threshold for such amino acids. Thus, the present invention may be useful in improving the taste of such products.

The inventive compositions are also expected to be useful in agrochemical products. Agrochemical products are delivered through a variety of delivery means, for example, solid forms, liquid forms, and aerosol forms. Efficacy often depends on the effective solubility of the active ingredients to ensure that proper dispersion of the active ingredient occurs, and in some cases, that the active ingredient is delivered at the desired time and location. For example, in certain herbicidal uses, a delayed release is often important to ensure that active ingredients are not released prior to the desired location. For example, if the herbicide needs to be released in the soil, a release in the air would be undesirable and result in limited or no efficacy. Conversely, if an early release is necessary, a low solubility could result in failure to deliver the active ingredient at the necessary time.

The inventive composition may be useful with the following agrochemical products: acylamino acid fungicides, acylamino acid fungicides, aliphatic amide organothiophosphate insecticides, aliphatic nitrogen fungicides, aliphatic organothiophosphate insecticides, amide fungicides, amide herbicides, anilide fungicides, anilide herbicides, antiauxins, antibiotic acaricides, antibiotic fungicides, antibiotic herbicides, antibiotic insecticides, antibiotic nematicides, aromatic acid herbicides, aromatic fungicides, arsenical herbicides, arsenical insecticides, arylalanine herbicides aryloxyphenoxypropionic herbicides, auxins, avermectin acaricides, avermectin insecticides, benzamide fungicides, benzanilide fungicides, benzimidazole fungicides, benzimidazole precursor fungicides, benzimidazolylcarbamate fungicides, benzofuranyl alkylsulfonate herbicides, benzofuranyl methylcarbamate insecticides, benzoic acid herbicides, benzothiazole fungicides, benzothiopyran organothiophosphate insecticides, benzotriazine organothiophosphate insecticides, benzoylcyclohexanedione herbicides, bipyridylium herbicides, botanical insecticides, botanical rodenticides, bridged diphenyl acaricides, bridged diphenyl fungicides, carbamate acaricides, carbamate fungicides, carbamate herbicides, carbamate insecticides, carbamate nematicides, carbanilate fungicides, carbanilate herbicides, chitin synthesis inhibitors, chloroacetanilide herbicides, chloronicotinyl insecticides chloropyridine herbicides, chlorotriazine herbicides, conazole fungicides, copper fungicides, coumarin rodenticides, cyclic dithiocarbamate fungicides, cyclodiene insecticides, cyclohexene oxime herbicides, cyclopropylisoxazole herbicides, cytokinins, defoliants, diacylhydrazine insecticides, dicarboximide fungicides, dicarboximide herbicides, dichlorophenyl dicarboximide fungicides, dimethylcarbamate insecticides, dinitroaniline herbicides, dinitrophenol acaricides, dinitrophenol fungicides, dinitrophenol herbicides, dinitrophenol insecticides, diphenyl ether herbicides, dithiocarbamate fungicides, dithiocarbamate herbicides, ethylene releasers, fluorine insecticides, formamidine acaricides, formamidine insecticides, fumigant insecticides, furamide fungicides, furanilide fungicides, gibberellins, growth inhibitors, growth retardants, growth stimulators, halogenated aliphatic herbicides, heterocyclic organothiophosphate insecticides, imidazole fungicides, imidazolinone herbicides, indandione rodenticides, inorganic fungicides, inorganic herbicides, inorganic insecticides, inorganic mercury fungicides, inorganic rodenticides, insect growth regulators, isoindole organothiophosphate insecticides, isoxazole organothiophosphate insecticides, juvenile hormone mimics, juvenile hormones, macrocyclic lactone acaricides, macrocyclic lactone insecticides, mercury fungicides, methoxytriazine herbicides, methylthiotriazine herbicides, milbemycin acaricides, milbemycin insecticides, mite growth regulators, morphactins, morpholine fungicides, moulting hormone agonists, moulting hormones, moulting inhibitors, nereistoxin analogue insecticides, nicotinoid insecticides, nitrile herbicides, nitroguanidine insecticides, nitromethylene insecticides, nitrophenyl ether herbicides, organochlorine acaricides, organochlorine insecticides, organochlorine rodenticides, organomercury fungicides, organophosphate acaricides, organophosphate insecticides, organophosphate nematicides, organophosphorus acaricides, organophosphorus fungicides, organophosphorus herbicides, organophosphorus insecticides, organophosphorus nematicides, organophosphorus rodenticides, organothiophosphate acaricides, organothiophosphate insecticides, organothiophosphate nematicides, organotin acaricides, organotin fungicides, oxadiazine insecticides, oxathiin fungicides, oxazole fungicides, oxime carbamate acaricides, oxime carbamate insecticides, oxime carbamate nematicides, oxime organothiophosphate insecticides, phenoxy herbicides, phenoxyacetic herbicides, phenoxybutyric herbicides, phenoxypropionic herbicides, phenyl ethylphosphonothioate insecticides, phenyl methylcarbamate insecticides, phenyl organothiophosphate insecticides, phenyl phenylphosphonothioate insecticides, phenylenediamine herbicides, phenylsulfamide acaricides, phenylsulfamide fungicides, phenylurea herbicides, phosphonate acaricides, phosphonate insecticides, phosphonothioate insecticides, phosphoramidate insecticides, phosphoramidothioate acaricides, phosphoramidothioate insecticides, phosphorodiamide acaricides, phosphorodiamide insecticides, phthalic acid herbicides, phthalimide acaricides, phthalimide fungicides, phthalimide insecticides, picolinic acid herbicides, polymeric dithiocarbamate fungicides, polysulfide fungicides, precocenes, pyrazole acaricides, pyrazole insecticides, pyrazolopyrimidine organothiophosphate insecticides, pyrazolyloxyacetophenone herbicides, pyrazolylphenyl herbicides, pyrethroid acaricides, pyrethroid ester acaricides, pyrethroid ester insecticides, pyrethroid ether acaricides, pyrethroid ether insecticides, pyrethroid insecticides, pyridazine herbicides, pyridazinone herbicides, pyridine fungicides, pyridine herbicides, pyridine organothiophosphate insecticides, pyridylmethylamine insecticides, pyrimidinamine acaricides, pyrimidinamine insecticides, pyrimidinamine rodenticides, pyrimidine fungicides, pyrimidine organothiophosphate insecticides, pyrimidinediamine herbicides, pyrimidinyloxybenzoic acid herbicides, pyrimidinylsulfonylurea herbicides, pyrimidinylthiobenzoic acid herbicides, pyrrole acaricides, pyrrole fungicides, pyrrole insecticides, quaternary ammonium herbicides, quinoline fungicides, quinolinecarboxylic acid herbicides, quinone fungicides, quinoxaline acaricides, quinoxaline fungicides, quinoxaline organothiophosphate insecticides, strobilurin fungicides, sulfite ester acaricides, sulfonanilide fungicides, sulfonanilide herbicides, sulfonylurea herbicides, tetronic acid acaricides, tetronic acid insecticides, thiadiazole organothiophosphate insecticides, thiadiazolylurea herbicides, thiazole fungicides, thiocarbamate acaricides, thiocarbamate fungicides, thiocarbamate herbicides, thiocarbonate herbicides, thiophene fungicides, thiourea acaricides, thiourea herbicides, thiourea rodenticides, triazine fungicides, triazine herbicides, triazinone herbicides, triazinylsulfonylurea herbicides, triazole fungicides, triazole herbicides, triazole organothiophosphate insecticides, triazolone herbicides, triazolopyrimidine herbicides, uracil herbicides, urea fungicides, urea herbicides, urea insecticides, urea rodenticides, valinamide fungicides, and acylamino acid fungicides.

The delivery system for such agrochemical products can be any conventional delivery system, including but not limited to solid powders, oils, liquids solubilized in aqueous or oil based solvents, aerosols, pellets, granules, pump sprays, tapes, films and suspensions.

The following examples set forth preferred embodiments of the present inventions. These embodiments are set forth for illustration purposes, and are not intended to limit the invention claimed herein.

General procedure: Zero to 40% w/v of various cyclodextrins were dissolved in 0.25% (w/v) PVP polymer, 50 mM MgCl₂ solutions with 50 to 150 mM of various amino acids, resulting in an aqueous complexation media. An excess amount of drug was added to the aqueous complexation media, with the suspension formed then sonicated for 60 minutes at 75° C. (some drugs can also be heated at 121° C. for 20 minutes in autoclave). After an equilibrium period, the drug suspensions were filtered and the amount of dissolved drug was determined by HPLC.

To provide further information as to a specific example, production of the third camptothecin formulation (with proline) listed below is described. 20% w/v of randomly methylated β-cyclodextrin was dissolved in 0.25% (w/v) CMC polymer, 50 mM MgCl₂ solutions with 50 mM of Proline and 0.02M HCl (buffer). The drug concentration was 0.526±0.009 mg/ml.

Camptotechin (CPT)

| | 20% RMβCD (mg/ml) | 40% HPβCD | 40% HPβCD |
|---|---|---|---|
| No amino acid | 0.228 ± 0.009 | | |
| Lysine | 0.285 ± 0.010 | 0.493 ± 0.015 | 0.439 ± 0.022 |
| Proline | 0.526 ± 0.009 | | |
| Cysteine | 0.334 ± 0.025 | | |
| Tryptophan | 0.375 ± 0.002 | | |

To provide further information as to a specific example, production of the first trichlocarban formulation (with lysine) listed below is described. 20% w/v of randomly methlyated β-cyclodextrin was dissolved in 0.25% (w/v) PVP polymer, 50 mM MgCl₂ solutions with 50 mM of L-Lysine hydrochloride. The drug concentration was 5.72±0.07 mg/ml.

Trichlocarban (TCC) solubility in mg/ml ± st.dev..

| | CD no | | CD + poly + | | CD + AA + | CD + pol + |
|---|---|---|---|---|---|---|
| 20% RMβCD | AA | CD + poly | metal ions | CD + AA | ions | ions + AA |
| Lysine | 2.05 ± 0.15 | 2.30 ± 0.24 | 2.61 ± 0.94 | 5.13 ± 0.10 | 5.42 ± 0.26 | 5.72 ± 0.07 |
| Leucine | 2.05 ± 0.15 | | | | | 4.79 ± 0.06 |
| Proline | 2.05 ± 0.15 | | 3.81 ± 0.20 | 5.20 ± 0.06 | 5.17 ± 0.12 | 5.33 ± 0.18 |
| Glutamic acid | 2.05 ± 0.15 | | | | | 4.67 ± 0.12 |
| Threonine | 2.05 ± 0.15 | | | | | 4.39 ± 0.07 |
| Tryptophan | 2.05 ± 0.15 | | | | | 4.66 ± 0.17 |
| Cysteine | 2.05 ± 0.15 | | | | | 5.08 ± 0.20 |

The solubility of TCC with adipic acid (amino acid analog) is 5.19±0.21 mg/ml and with poly-lysine it is 4.81±0.21.

[image]

[image]

What is claimed is:

1. A composition comprising cyclodextrin and one or more amino acids, wherein said cyclodextrin is selected from the group of cyclodextrins consisting of natural cyclodextrins and alkylated cyclodextrins, and said amino acid is selected from the group of amino acids consisting of all amino acids, derivatives thereof and homo- or co-polymers of said amino acids, the ratio of cyclodextrin to amino acid being in the range of 50:1 to 1:1 cyclodextrin:amino acid.

2. The composition of claim 1 wherein said cyclodextrin is selected from the group consisting of natural cyclodextrins and alkylated cyclodextrins.

3. The composition of claim 2 wherein said cyclodextrin is selected from the group consisting of methyl β-cyclodextrin, dimethyl β-cyclodextrin, trimethyl β-cyclodextrin, randomly methylated β-cyclodextrin, randomly methylated a-cyclodextrin, randomly methylated d-cyclodextrin, a, β, d-cyclodextrins, and other alkylated cyclodextrins.

4. The composition of claim 1 wherein said amino acid is selected from one or more of the group consisting of all natural amino acids, including all isomeric forms individually and in racemic and non-racemic mixtures, and analogs of amino acids, including all isomeric forms individually and in racemic and non-racemic mixtures, and further including mixtures of each of the above.

5. The composition of claim 4 wherein said amino acid is selected from the group consisting of Alanine, Isoleucine, Leucine, Methionine, Phenylalanine, Proline, Tryptophan, Valine, Asparagine, Cysteine, Glutamine, Glycine, Serine, Threonine, Tyrosine, Aspartic Acid, Glutamic Acid, Arginine, Hystidine, and Lysine, including all isomeric forms individually and in racemic and non-racemic mixtures and further including mixtures of each of the above.

6. The composition of claim 1 wherein said composition further comprises metal ion in a molar concentration of from about 10 mM to about 200 mM.

7. The composition of claim 1 wherein said metal ion is selected from the group consisting of magnesium, iron and zinc ions.

8. The use of the composition of claim 1 to form a complex comprising ingredients selected from the group comprising active pharmaceutical compounds, foods, beverages, nutritional products, cosmetics, and agrochemicals wherein said ingredient is combined with said composition of claim 1 to form a complex whereby said ingredient has improved solubility compared to a composition in which no amino acid, derivatives thereof or homo- or co-polymers are used.

9. The use of claim 8 wherein said complex further comprises a metal ion in a molar concentration of from about 10 mM to about 200 mM.

10. A composition comprising a cyclodextrin selected from the group consisting of natural cyclodextrins and alkylated cyclodextrins, one or more amino acids, analogs, derivatives thereof or homo- or copolymer of said amino acids, the ratio of cyclodextrin to amino acid being in the range of 50:1 to 1:1 cyclodextrin:amino acid, and an ingredient selected from the group of active pharmaceutical ingredients, foods, beverages, nutritional products, cosmetics, and agrochemicals.

11. The composition of claim 10 further comprising a metal ion in a molar concentration of from about 10 mM to about 200 mM.

12. The composition of claim 8 wherein said composition includes an ingredient selected from the group of active pharmaceutical ingredients and is included in a delivery form selected from the group comprising transdermal and dermal patches and creams, eye drops, syrups such as cough syrups, mouthwash, toothpaste, cosmetics, soaps, pump sprays, sublingual tablets, sublingual films, quick-dissolve tablets, quick-dissolve films, chewing gums, lozenges, nanocrystals, and detergents with active ingredients.

13. The composition of claim 8 wherein said composition includes an ingredient selected from the group of agrochemicals and is included in a delivery form selected from the group comprising solid powders, oils, liquids solubilized in aqueous or oil based solvents, aerosols, pellets, granules, pump sprays, tapes, films and suspensions.

14. A method for delivering active pharmaceutical ingredients via non-parenteral dosage forms, comprising the steps of (a) forming a non-inclusion complex comprising said active pharmaceutical ingredient, a cyclodextrin selected from the group consisting of natural cyclodextrins and alkylated cyclodextrins, and one or more amino acids selected from the group of amino acids consisting of all amino acids, analogs, derivatives thereof and homo- or co-polymers of said amino acids, the ratio of cyclodextrin to amino acid being in the range of 50:1 to 1:1 cyclodextrin:amino acid and the amount of said active pharmaceutical ingredient being in the range of 1:1 to 1:40 active ingredient: cyclodextrin, and (b) administering said non-inclusion complex to a human or animal subject by a non-parenteral dosage form selected from the group of dosage forms consisting of tablets, intravenous or oral solutions, intravenous or oral suspensions, dry powder, nasal or oral spray, patches, eye or ear drops, cream, mouthwash or toothpaste.

15. A method for enhancing the complexation of a composition comprising cyclodextrin, one or more amino acids, and an ingredient selected from the group of active pharmaceutical ingredients, foods, beverages, nutritional products, cosmetics, and agrochemicals, comprising the step of sonicating said composition for about 10 minutes to 60 minutes at about 60 to 80° C.

16. A method for enhancing the complexation of a composition comprising cyclodextrin, one or more amino acids, and an ingredient selected from the group of active pharmaceutical ingredients, foods, beverages, nutritional products, cosmetics, and agrochemicals, comprising the step of autoclaving said composition for about 5 minutes to 30 minutes at 110 to 130° C.
